(19) United States
(12) Patent Application Publication
(10) Pub. No.: US 2025/0152959 A1
(43) Pub. Date: May 15, 2025

(54) CATHETER INCLUDING A FOAM GENERATION ASSEMBLY

(71) Applicant: Solv Endotherapy Inc., Baltimore, MD (US)

(72) Inventors: Anthony APPLING, Crestwood, KY (US); Benjamin MORRIS, Jeffersonville, IN (US)

(21) Appl. No.: 18/947,726

(22) Filed: Nov. 14, 2024

Related U.S. Application Data
(60) Provisional application No. 63/548,416, filed on Nov. 14, 2023.
(60) Provisional application No. 63/603,162, filed on Nov. 28, 2023.

(51) Int. Cl. A61N 2/02; A61N 2/00

(52) U.S. Cl. CPC A61N 2/02; A61N 2/006

(57) ABSTRACT

A catheter includes a catheter shaft extending from a proximal catheter end to a distal catheter end. A plurality of lumens are housed with the catheter shaft and each configured to separately convey respective substances, such as a drug/therapy solution in a first lumen and a gas in a second lumen, from a proximal lumen end disposed adjacent the proximal catheter end to a distal lumen end. A foam generation assembly extends from an entrance end fluidly connected to the distal lumen ends for receiving the respective substances to an exit end disposed adjacent or in spaced relationship with the distal catheter end of the catheter shaft. The foam generation assembly includes a plurality of shearing structures arranged between the entrance and exit ends for mixing the respective substances as they are conveyed through the foam generation assembly to generate a foam at the exit end.

### CROSS-REFERENCE TO RELATED APPLICATIONS

The subject patent application claims priority to U.S. Provisional Application Ser. No. 63/548,416 filed on Nov. 14, 2023 and U.S. Provisional Application Ser. No. 63/603,162 filed on Nov. 28, 2023.

### BACKGROUND OF THE INVENTION

### 1. Field of the Invention

The present disclosure is generally related to a medical device. More particularly, the present disclosure relates to a catheter for delivering and transitioning a drug/therapy solution into a foam at a distal end.

### 2. Related Art

This section provides background information related to the present disclosure which is not necessarily prior art.

A catheter is a medical instrument for use in accessing a target site inside of a patient's body. The catheter includes a catheter shaft that extends from a proximal end to a distal end, and defines at least one lumen to deliver a drug/therapy solution to the distal end. The drug/therapy solution commonly exits the distal end for application over a surface of the target site (e.g., a three-dimensional anatomical cavity). However, in application, without adhesive properties of the drug/therapy solution over such surface, the drug/therapy solution will tend to flow to the lowest point due to gravity, and thus is not effectively applied to treat the entire surface of the three-dimensional anatomical cavity. Accordingly, it is known in the prior art catheters to generate a foam from the drug/therapy solution as it exits the distal end to prolong an exposure of the drug/therapy solution to the surface of the target site. One such example is disclosed in WO 2023/028251 to The John Hopkins University. However, such current assemblies and related methods of foam generation at the distal end of the catheter are still not optimized to produce foam with sufficient quantity, density and/or stability to effectively expose the target site to the drug/therapy solution for treatment.

Accordingly, there remains a continuing need for a catheter that improves the generation of foam with the proper characteristics depending on the target site and/or the drug/therapy solution.

### SUMMARY OF THE INVENTION

This section provides a general summary of the disclosure and is not intended to be a comprehensive disclosure of its full scope, aspects, objectives, and/or all of its features.

The subject disclosure is generally directed to a catheter capable of delivering a drug solution to a distal catheter end disposed adjacent a target site, and generating a controlled volume of foam at the distal catheter end to fill a cavity and saturate a surface area of the target site with the drug/therapy solution over the duration of the generated foam. In a preferred arrangement, the catheter includes a catheter shaft that extends along a longitudinal axis from a proximal catheter end to a distal catheter end. A plurality of lumens are housed with the catheter shaft and each configured to separately convey respective substances, such as a drug/therapy solution in a first lumen and a gas in a second lumen, from a proximal lumen end disposed adjacent the proximal catheter end to a distal lumen end. A foam generation assembly extends from an entrance end fluidly connected to the distal lumen ends of the plurality of lumens for receiving the respective substances to an exit end disposed adjacent or in spaced relationship with the distal catheter end of the catheter shaft. The foam generation assembly includes a plurality of shearing structures arranged between the entrance and exit ends for mixing the respective substances as they are conveyed through the foam generation assembly to generate a foam as the mixed substances exit the foam generation assembly at the exit end.

The presence of the plurality of shearing structures in the foam generation assembly provides an improved means for generating foam with increased quantities, densities, and stability that resist gravity due to the improved supporting foam structure. More specifically, the plurality of shearing structures result in increased efficiency of mixing of the substances due to creating gross shear of the first substance (e.g., drug/therapy solution) with the second substance (e.g., gas) by the plurality of shearing structures, resulting in the creation of an improved foam structure (e.g., micro bubble foam) that has improved density and stability to provide increased and extended exposure of the incorporated drug/therapy solution at the target site. In summary, the foam generation assembly provides improved foam generation over the prior art assemblies and methods, which results in improved treatment of the target site with the incorporated drug/therapy solution.

### BRIEF DESCRIPTION OF THE DRAWINGS

Other aspects of the present disclosure will be readily appreciated, as the same becomes better understood by reference to the following detailed description when considered in connection with the accompanying drawings wherein:

FIG. 1 is a perspective view of a catheter including a catheter shaft extending from a proximal catheter end to a distal catheter end, a plurality of lumens extending within the catheter shaft from a proximal lumen end disposed adjacent the proximal catheter end to a distal lumen end disposed adjacent the distal catheter end, and a foam generation assembly disposed at the distal catheter end in fluid communication with the distal lumen ends of the plurality of lumens;

FIG. 2 is a perspective view of an alternative arrangement of the catheter illustrating the distal lumen ends terminating in spaced relationship with the distal catheter end to define a mixing chamber therebetween, and the foam generation assembly disposed within the catheter shaft in spaced relationship with the distal catheter end;

FIG. 3A is a cross-sectional view of the catheter shaft taken along 3A-3A of FIGS. 1 and 2 illustrating the plurality of lumens including a first lumen and a second lumen;

FIG. 3B is a cross-sectional view of the catheter shaft taken along 3B-3B of FIG. 2 illustrating the mixing chamber being absent the plurality of lumens;

FIG. 4 is a perspective cross-sectional view of the catheter shaft more clearly illustrating the first and second lumens of the plurality of lumens;

FIGS. 5-8 are perspective cross-sectional views of the catheter shaft illustrating alternative arrangements of the plurality of lumens;

FIG. 9 is a cross-sectional side view illustrating a catheter hub arranged at the proximal catheter end of the catheter shaft and defining a first port for introducing a first substance (e.g., a fluid drug/therapy solution) to the first lumen of the plurality of lumens and a second port for introducing a second substance (e.g., gas) to the second lumen of the plurality of lumens;

FIG. 10 is a side view of a first embodiment of the foam generation assembly arranged on the distal catheter end of the catheter shaft and having an entrance end disposed in fluid communication with the distal lumen ends of the first and second lumens for receiving and mixing the first and second substances to generate a foam;

FIG. 11 is a cross-sectional perspective view of FIG. 10 to more clearly illustrate the first embodiment of the foam generation assembly being an open cell foam body and having a polygonal cross-sectional shape;

FIGS. 12A-12D are perspective views of the first embodiment of the foam generation assembly illustrating various geometries and shapes for the open cell foam body;

FIG. 13 is a fragmentary side cross-sectional view of the catheter shaft illustrating the first embodiment of the foam generation assembly alternatively arranged within the catheter shaft in spaced relationship with the distal catheter end and having a cylindrical cross-sectional shape;

FIG. 14 is the fragmentary side cross-sectional view of FIG. 13 additionally illustrating the foam generation assembly in cross-section to more clearly illustrate the open cell foam body;

FIG. 15 is a side view of a second embodiment of the foam generation assembly comprised of at least one layer of porous woven material;

FIG. 16 is a perspective cross-sectional view of the second embodiment of the foam generation assembly to more clearly illustrate the porous woven material;

FIG. 17 is a side view of a third embodiment of the foam generation assembly comprised of at least one layer of fibrous woven material;

FIG. 18 is a perspective cross-sectional view of the third embodiment of the foam generation assembly to to more clearly illustrate the fibrous woven material;

FIG. 19 is a perspective view of a fourth embodiment of the foam generation assembly including at least one mesh disc arranged transverse to a longitudinal axis of the catheter shaft;

FIG. 20 is a cross-sectional perspective view of the fourth embodiment of the foam generation assembly;

FIG. 21 is a perspective view of an alternative arrangement of the fourth embodiment of the foam generation assembly;

FIG. 22 is a cross-sectional perspective view of FIG. 21 illustrating the at least one mesh disc including an initial mesh disc arranged adjacent the entrance end of the foam generation assembly and a last mesh disc arranged adjacent the exit end of the foam generation assembly;

FIG. 23 is a cross-sectional perspective view of the fourth embodiment of the foam generation assembly illustrating the at least one mesh disc including a plurality of mesh discs sequentially arranged between the entrance and exit ends;

FIG. 24 is a side cross-sectional view of the fourth embodiment shown in FIG. 23;

FIG. 25 is a perspective view of a fifth embodiment of the foam generation assembly;

FIG. 26 is a perspective view of the fifth embodiment of the foam generation assembly illustrating a rolled mesh which is rolled concentrically around the longitudinal axis of the catheter shaft;

FIG. 27 is a cross-sectional perspective view of the fifth embodiment of the foam generation assembly;

FIG. 28 is a perspective view of a sixth embodiment of the foam generation assembly including a three-dimensional filter tip disposed adjacent the exit end;

FIG. 29 is a cross-sectional perspective view of the sixth embodiment of the foam generation assembly illustrating a mesh disc disposed adjacent the entrance end;

FIGS. 30A-F are perspective and side views of the sixth embodiment of the foam generation assembly illustrating various shapes and geometries for the three-dimensional filter tip;

FIG. 31 is a fragmentary perspective view of the sixth embodiment of the foam generation assembly illustrating the three-dimensional filter tip in cross-section to more clearly illustrate at least one strut supporting and defining the three-dimensional profile;

FIG. 32 is a perspective view of a seventh embodiment of the foam generation assembly illustrating a paneled filter tip disposed adjacent the exit end;

FIG. 33 is a cross-sectional perspective view of the seventh embodiment of the foam generation assembly illustrating a mesh disc disposed adjacent the entrance end;

FIG. 34 is a perspective view of an eighth embodiment of the foam generation assembly including an impeller extending between the entrance and exit ends and rotatable about the longitudinal axis of the catheter shaft;

FIG. 35 is a perspective view of the eighth embodiment of the foam generation assembly with a housing shown in phantom to more clearly illustrate the impeller being threaded between the entrance and exit ends;

FIG. 36 is a fragmentary perspective view of the eight embodiment of the foam generation assembly;

FIG. 37 is a perspective view of the eighth embodiment of the foam generation assembly illustrating an alternative arrangement in which the impeller is disposed in spaced relationship with the distal catheter end;

FIG. 38 is a perspective view of the eighth embodiment of the foam generation assembly shown in FIG. 37 with the housing shown in phantom to more clearly illustrate a mixing chamber disposed between the exit end of the impeller and the distal catheter end;

FIG. 39 is a cross-sectional view of the eighth embodiment of the foam generation assembly shown in FIG. 37;

FIG. 40 is a perspective view of the eighth embodiment of the foam generation assembly with the housing shown in phantom to illustrate an alternative arrangement of the impeller comprised of an eccentric wire form being non-symmetrical relative to the longitudinal axis; and

FIG. 41 is a side view of an alternative arrangement of the eccentric wire having multiple bends arranged offset relative to the longitudinal axis.

### DETAILED DESCRIPTION OF THE ENABLING EMBODIMENTS

Example embodiments will now be described more fully with reference to the accompanying drawings. The example embodiments are provided so that this disclosure will be thorough and fully convey the scope to those skilled in the art. In some example embodiments, well-known processes, well-known device structures, and well-known technologies are not described in detail. Numerous specific details are set forth such as examples of specific components, devices, mechanisms, assemblies, and methods to provide a thorough understanding of various embodiments of the present disclosure. It will be apparent to those skilled in the art that specific details need not be employed, that example embodiments may be embodied in many different forms, and that neither should be construed to limit the scope of the disclosure. With this in mind, the present disclosure is generally directed to a catheter capable of delivering a drug/therapy solution to a target site and generating a controlled volume of foam from the drug/therapy solution, to fill a cavity and saturate a surface area of the target site with the drug/therapy solution over the duration of the generated foam.

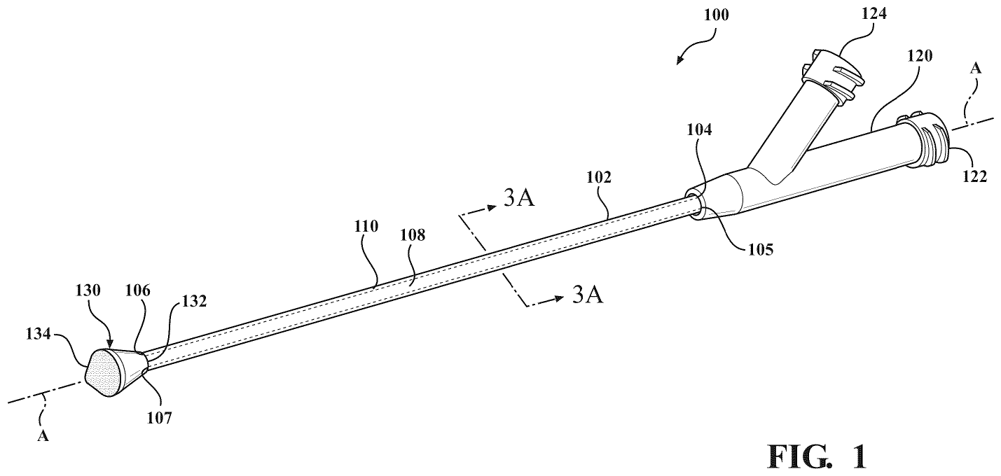

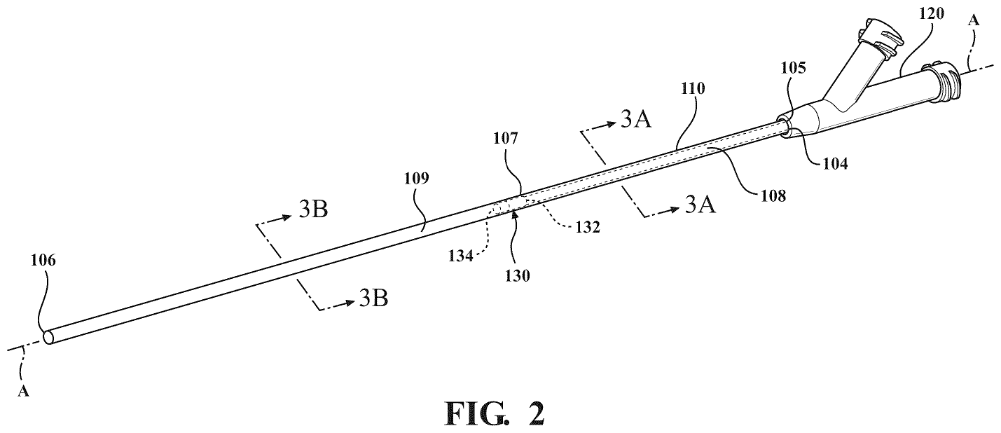

As best illustrated in FIGS. 1-2, the catheter 100 includes a catheter shaft 102 extending from a proximal catheter end 104 to a distal catheter end 106 and which preferably houses or defines a plurality of lumens 108, 110 each extending adjacent and separately from one another from a proximal lumen end 105 disposed adjacent the proximal catheter end 104 to a distal lumen end 107 disposed either adjacent (FIG. 1) or in spaced relationship from (FIG. 2) the distal catheter end 106. Put another way, the plurality of lumens 108, 110 either extend along the entire catheter shaft 102 or terminate short of and in spaced relationship with the distal catheter end 106 to define an open and undivided mixing chamber 109 extending between the distal lumen end 107 of the plurality of lumens 108, 110 and the distal catheter end 106 of the catheter shaft 102. In either arrangement, the plurality of lumens 108, 110 are configured to separately convey respective substances, such as distinct drug solutions and/or gases, from the proximal lumen end 105 to the distal lumen end 107 such that the substances are isolated from one another within the plurality of lumens 108, 110 as they travel from the proximal lumen end 105 to the distal lumen end 107.

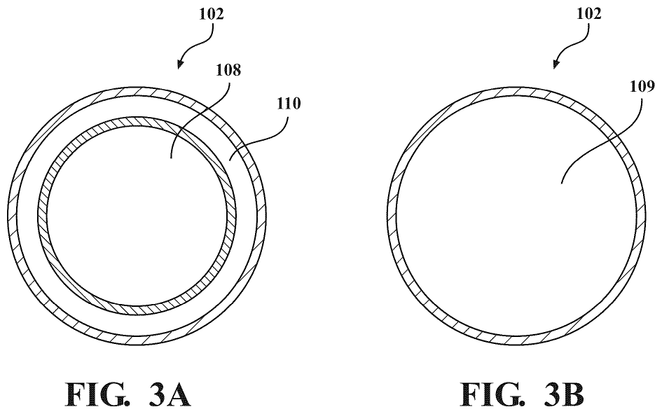

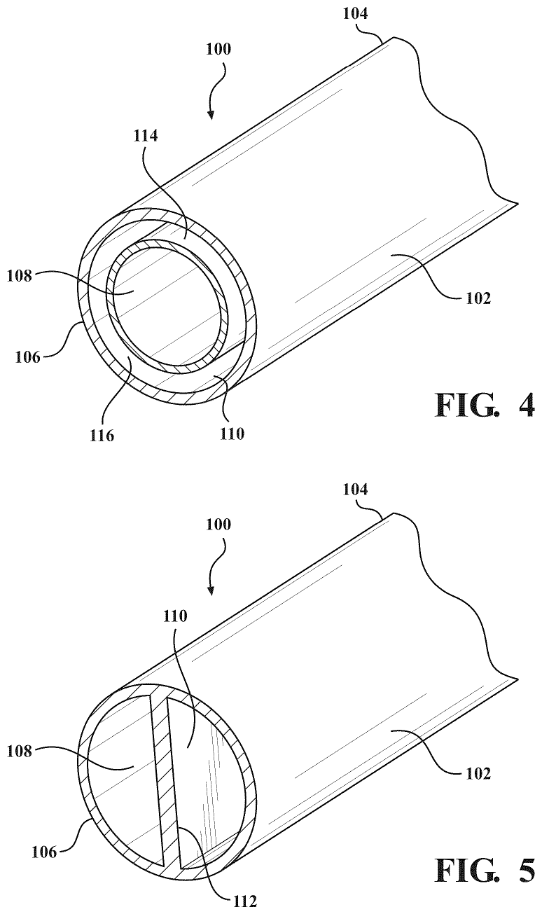

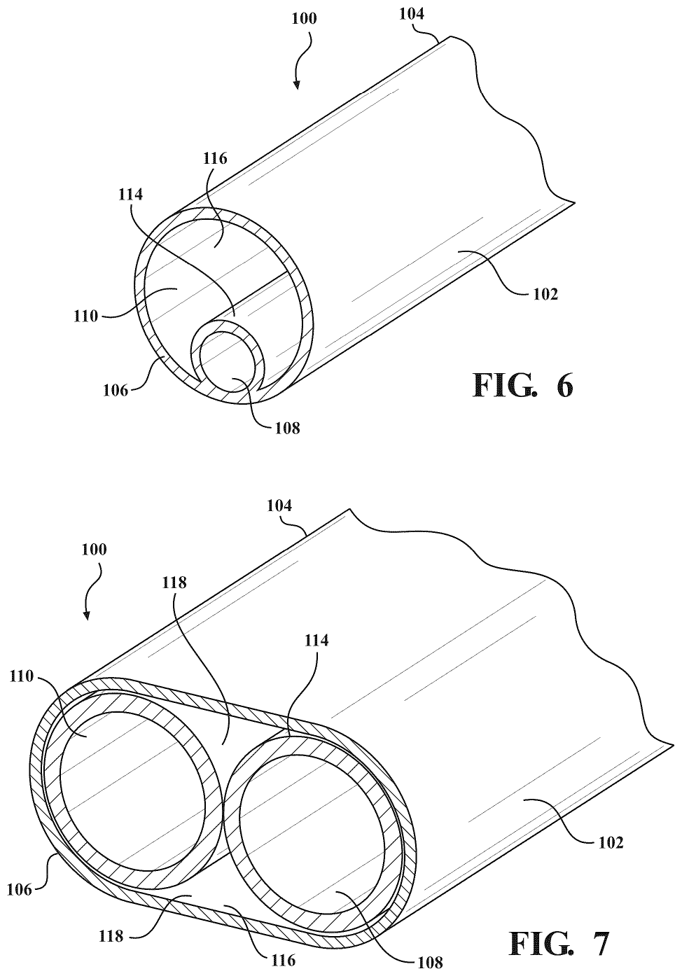

As best illustrated in FIGS. 3A and 4-7, in a preferred arrangement, the plurality of lumens 108, 110 include a pair of lumens 108, 110 extending between the proximal and distal lumen ends 105, 107, with a first lumen 108 arranged to receive and convey a first fluid or gas substance (e.g., a drug solution) and a second lumen 110 arranged to receive and convey a second fluid or gas substance (e.g., gas, such as CO₂) that is different from the first substance. As illustrated in FIG. 5, in a first arrangement the catheter shaft 102 can include a bifurcation 112 that extends between the proximal and distal lumen ends 105, 107 to define and separate the first and second lumens 108, 110 within the catheter shaft 102 on opposing and adjacent sides. Alternatively, in a second arrangement, the first lumen 108 can be arranged within the catheter shaft 102 either concentrically/co-axially (FIGS. 3A and 4) or eccentrically (FIG. 6) to define the second lumen 110 as the area extending radially between an outer wall 114 of the first lumen 110 and an inner wall 116 of the catheter shaft 102. Further, as shown in FIG. 7, in a third arrangement, the first lumen 108 and the second lumen 110 may each be separate structures from one another that are disposed within the catheter shaft 102 (e.g., the first lumen 108 and the second lumen 110 may be structurally distinct tubular shafts, or the like). In this arrangement, the orientation of the first lumen 108 and the second lumen 110 may form at least one working gap 118 between either of the first lumen 108 or the second lumen 110 and the catheter shaft 102, through which equipment such as support structures, wires, sensors, or the like, and/or other substances may be conveyed.

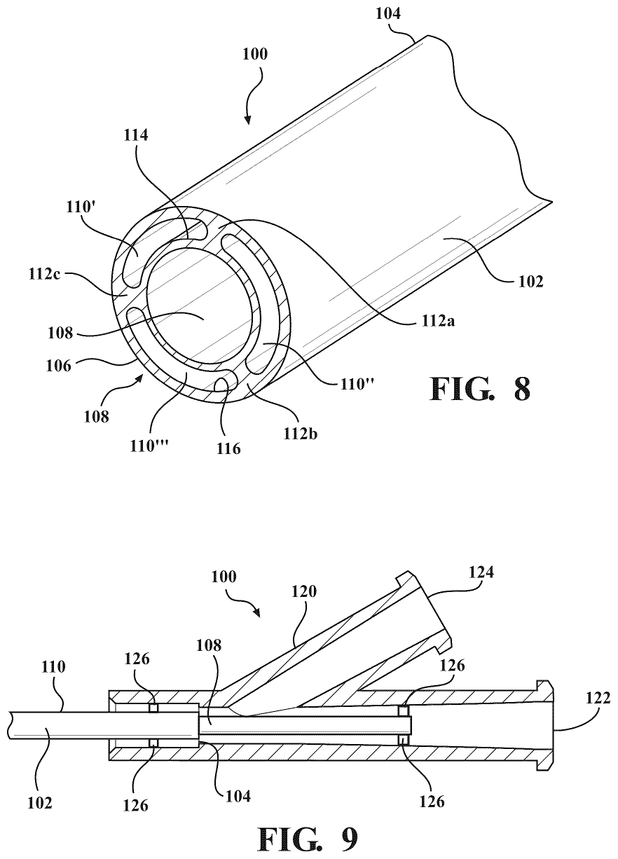

In yet another arrangement, more than a pair of lumens 108, 110 can be utilized for the plurality of lumens without departing from the scope of the subject disclosure. For example, as illustrated in FIG. 8, the first lumen 108 can be arranged concentrically/coaxially within the catheter shaft 102 and a plurality of second (or secondary) lumens 110′, 110″, 110′″ can be disposed between the inner wall 116 of the catheter shaft 102 and the outer wall 114 of the first lumen 110. A plurality of bifurcations 112a, 112b, 112c may be used to separate the plurality of secondary lumens 110′, 110″, 110″ arranged concentrically around the first lumen 108, and if present provide additional support for the structure of the first lumen 108. Although not expressly illustrated, the first lumen 108 may be disposed off-center within the catheter shaft 102, without departing from the scope of this arrangement.

As illustrated in FIGS. 1-2 and 9, in any of the aforementioned arrangements, the catheter 100 may include a catheter hub 120 arranged at the proximal catheter end 104 of the catheter shaft 102 and disposed in fluid communication with the proximal lumen end 105 of the plurality of lumens 108, 110. The catheter hub 120 defines a first port 122 disposed in fluid communication with the first lumen 108 for use in introducing the first substance (e.g., the fluid drug/therapy solution) to the first lumen 108 and a second port 124 disposed in fluid communication with the second lumen 110 for use in introducing the second substance (e.g., the CO₂ gas) to the second lumen 110. The catheter hub 120 can include a plurality of seals 126 for establishing a fluid-tight seal within the catheter hub 102 to ensure that substances introduced into the first port 122 and the first lumen 108 do not prematurely reach and mix with substances introduced in the second port 124 and the second lumen 110. As described previously, after the first and second substances are introduced into their respective lumens 108, 110, they travel separately through the lumens 108, 110 in isolated relationship from one another until they reach the distal lumen end 107, either disposed adjacent or in spaced relationship with the distal catheter end 106 of the catheter shaft 102.

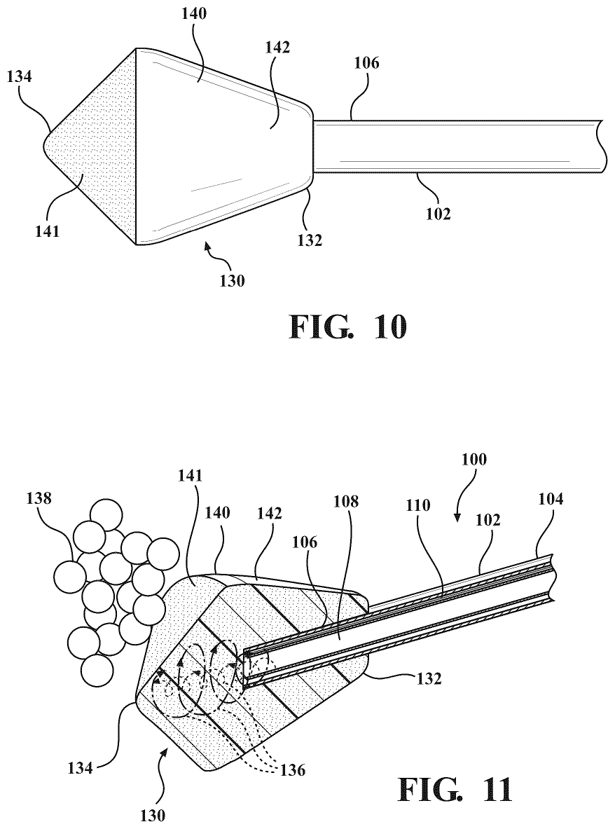

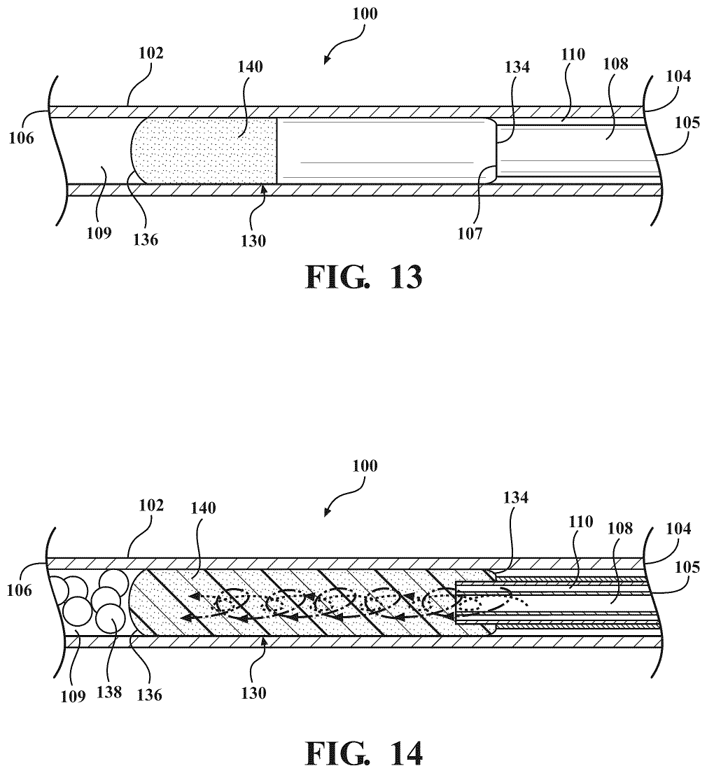

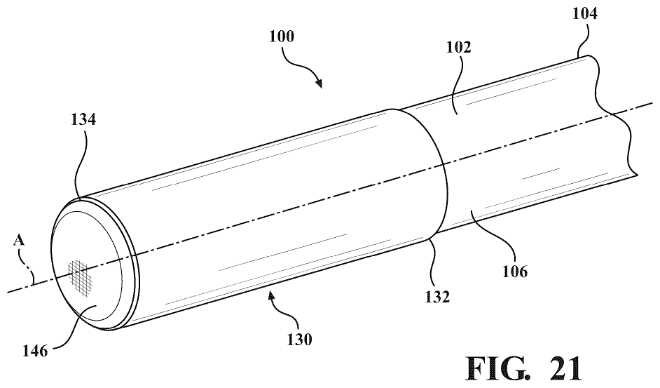

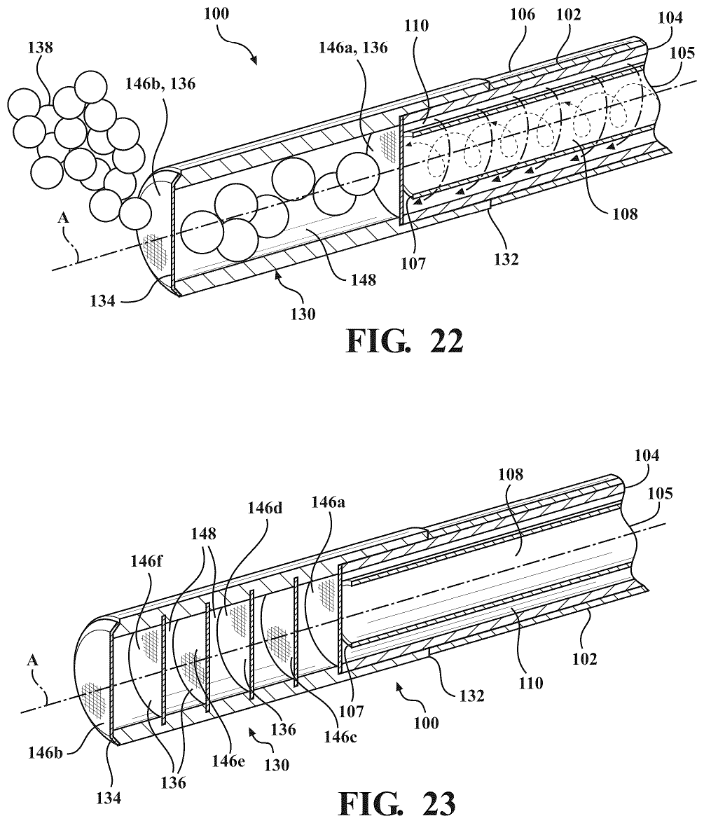

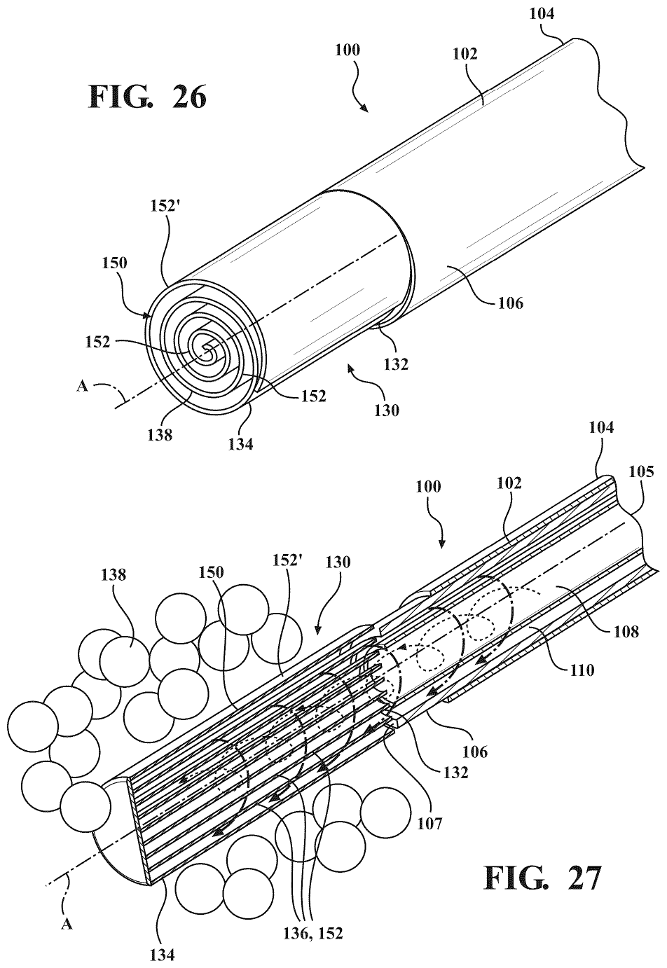

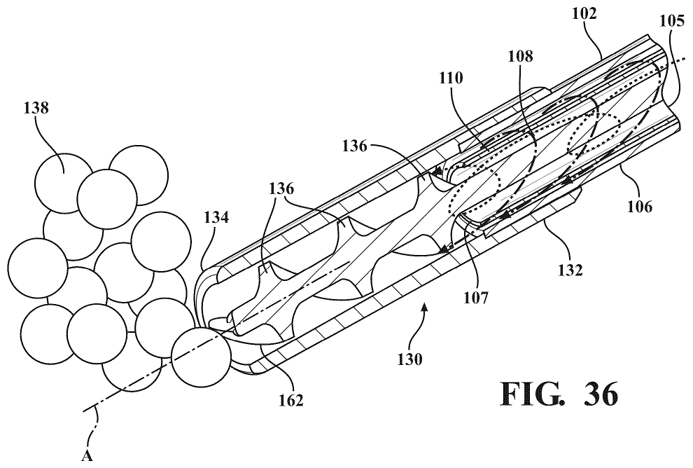

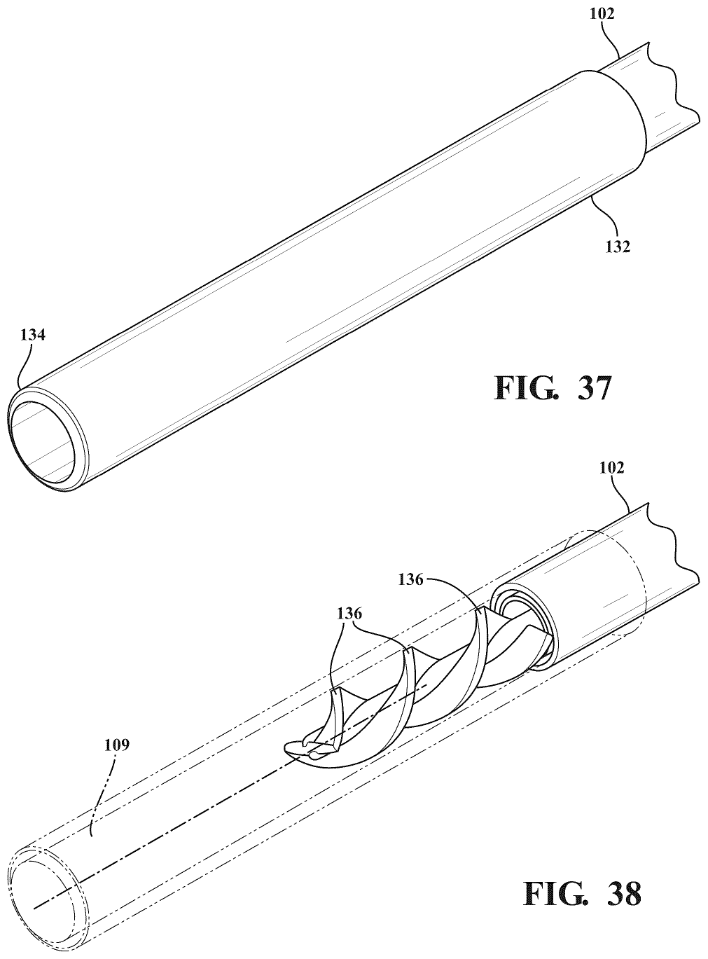

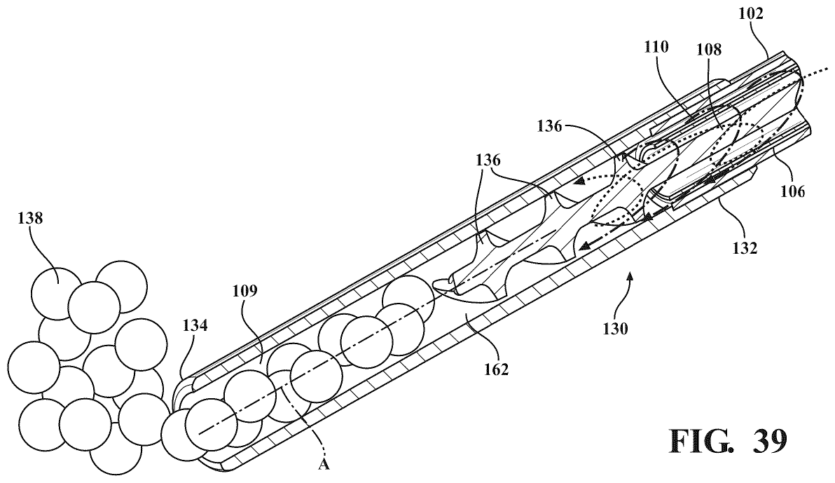

As best illustrated in FIGS. 1-2 and 10-40, the catheter 100 includes a foam generation assembly 130 arranged either adjacent or in spaced relationship with the distal catheter end 106 of the catheter shaft 102 and extending from an entrance end 132 fluidly connected to the distal lumen ends 107 of the plurality of lumens 108, 110 to an exit end 134, either disposed in fluid communication with an environment of the catheter shaft 102 or in fluid communication with the mixing chamber 109 (e.g., FIGS. 2, 13-14 and 38-19). In either arrangement, the foam generation assembly 130 includes a plurality of shearing structures 136 which are sequentially and/or uniformly arranged between the entrance and exit ends 132, 134 for mixing the substances received from the lumens 108, 110 as they flow and are conveyed through the foam generation assembly 130 to generate a foam 138 (See e.g., FIGS. 11, 14, 16, 18, 20, 22, 27, 31, 33, 36 and 39-40) at the exit end 134. The plurality of shearing structures 136 are configured and arranged to create gross shear of the first substance (e.g., the liquid drug solution) while mixing with the second substance (e.g., the CO₂ gas) to provide a means for generating a micro bubble foam 138 with increased quantities, densities, and stability for the resultant foam structure than possible with the prior art foam generation constructs. In other words, the geometries, lattices, arrangements, materials, porosities, or the like, in the various embodiments of the shearing structures 136 provide an overall gross shear that generates an improved foam structure 138 at the exit end 134 of the foam generation assembly 130 that is more resistant to gravity, and thus provides an extended exposure time of the drug/therapy solution to the surface of the target site, resulting in a more effective treatment than achievable by the prior art foams. Furthermore, when the foam generation assembly 130 is disposed in spaced relationship with the distal catheter end 106, the foam 138 is introduced into the mixing chamber 109 which provides for additional mixing of the foam 138 as it travels within the mixing chamber 109 from the exit end 134 of the foam generation assembly 130 to the distal catheter end 106. This additional length of travel allows the foam 138 to become thicker and with a more viscous consistency, like shaving cream.

As illustrated in FIGS. 1-2 and 10-14, in accordance with a first embodiment, the foam generation assembly 130 is comprised of an open cell foam body 140 (i.e., a porous, sponge like component) that defines the plurality of shearing structures 136 (best shown in the cross-sectional views of FIGS. 11 and 14) as being a lattice of interconnected cells/pores within the open cell foam body 140. The first and second substances flow from the plurality of lumens 108, 110 and through interconnected cells of the open cell foam body 140 for mixing and generating the foam 138 at and out of a “large” front surface area 141 disposed adjacent the exit end 134 of the foam generation assembly 130. In other words, ingress of the drug solution (as the first substance) into the open cell foam body 140 results in gross shear, while the open cell foam body 140 also accommodates ingress of gas (as the second substance) to mix within the open cell foam body 140 (the mixing shown by the semi-circular arrows in FIGS. 11 and 14) to result in the generation of foam 138 out of the front surface area 141 disposed adjacent the exit end 132.

The open cell foam body 140 is advantageously pliable such that when the foam generation assembly 130 is disposed adjacent and arranged on the distal catheter end 102, the open cell foam body 140 can be compressed when passing through a restricted channel such as an endoscope instrument channel or other means of delivering the distal catheter end 106 of the catheter shaft 102. After being compressed the foam generation assembly 130 can expand back to its original size or shape. As best illustrated in FIGS. 1 and 10-11, when the open cell foam body 140 is disposed on the distal catheter end 102, the open cell foam body 140 can have a polygonal cross-sectional shape to present a rear outer surface 142 of the open cell foam body 140 disposed adjacent the entrance end 132 which may be non-porous or respectively less-porous than a front outer surface 141 such that flow of the generated foam 138 is directed towards and out of the exit end 134. Alternatively, as best illustrated in FIGS. 2 and 13-14, when the foam generation assembly 130 is disposed within the catheter shaft 102 in spaced relationship with the distal catheter end 106, the open cell foam body 140 can have a cylindrical cross-sectional shape.

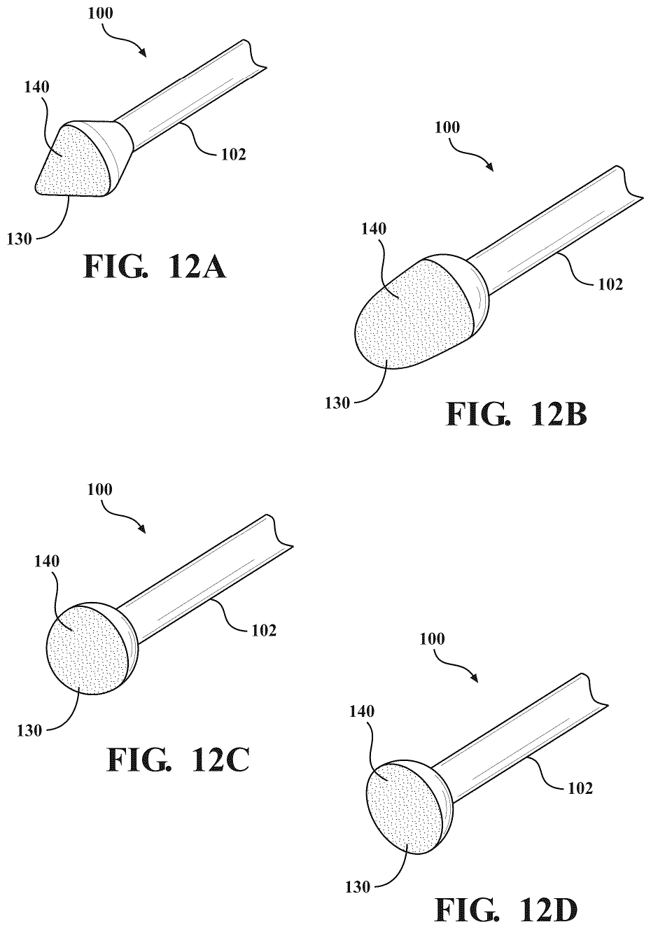

The foam generation assembly 130 may be fabricated by various means such as converting, molding, or the like or a combination or sub-combination thereof. Further, as best illustrated in FIGS. 12A-12D, the foam generation assembly 140 can have various other geometries and shapes, such as oval shaped (FIG. 12B), circular shaped (FIG. 12C) or semi-circular shaped (FIG. 12D), depending on the intended application and the restricted channel through which the open cell foam body 140 must pass.

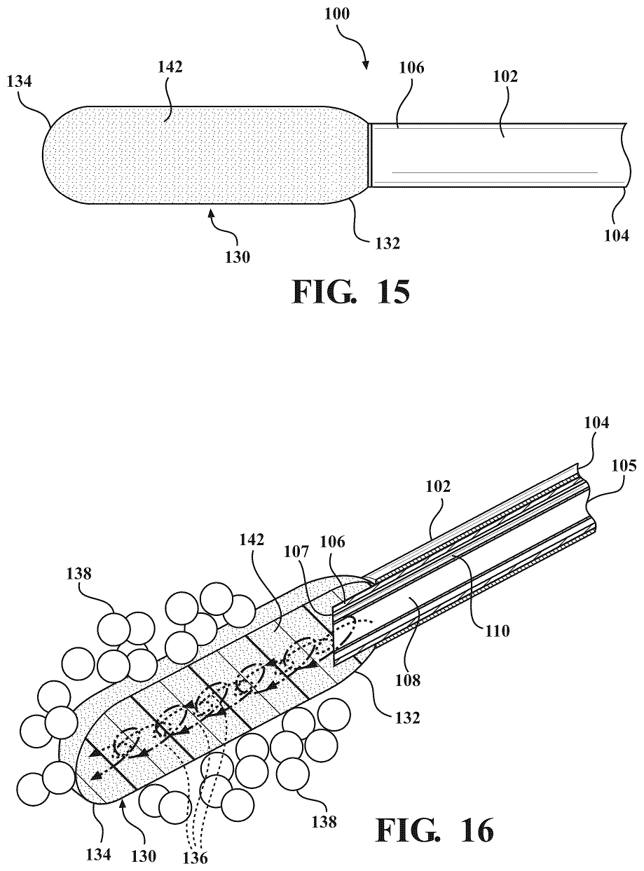

As illustrated in FIGS. 15-16, in accordance with a second embodiment, the foam generation assembly 130 is comprised of single or multiple layers of porous woven material, fiber, or fabric 142 which defines the plurality of shearing structure 136 as being a lattice of sequentially arranged pores (best shown in the cross-sectional view of FIG. 16). The first and second substances received from the plurality of lumens 108, 110 are forced through the porous material, creating shear and mixing the first substance (e.g., the drug/solution) with the second substance (e.g., gas) to generate gross foam at the “large” surface area adjacent the exit end 134. Similar to the open cell foam body of the first embodiment, the woven material of the second embodiment may also be pliable such that the foam generation assembly 130 may be compressed when passing through a restricted channel such as an endoscope instrument channel or other means of delivering the catheter 100. After being compressed the foam generation assembly 130 may expand to its original size or shape.

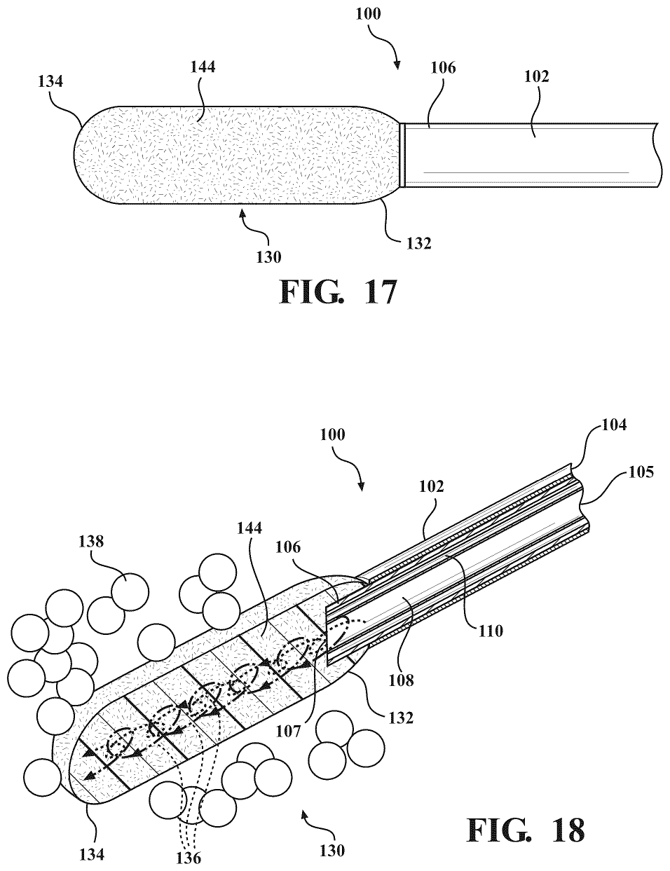

As illustrated in FIGS. 17-18, in accordance with a third embodiment, the foam generation assembly 130 is comprised of a single or multiple layer of fibrous woven material 144, which defines the plurality of shearing structure 136 as being a lattice of sequentially arranged fibers (best shown in the cross-sectional view of FIG. 18). The first and second substances received from the lumens 108, 110 are forced through the fibrous material, creating shear and mixing the first substance (e.g., the drug/solution) with the second substance (e.g., gas) to generate gross foam at the “large” surface area adjacent the exit end 134. Similar to the first and second embodiments, the fibrous material of the third embodiment may also be pliable such that the foam generation assembly 130 may be compressed when passing through a restricted channel such as an endoscope instrument channel or other means of delivering the catheter 100. After being compressed the foam generation assembly 130 may expand to its original size or shape.

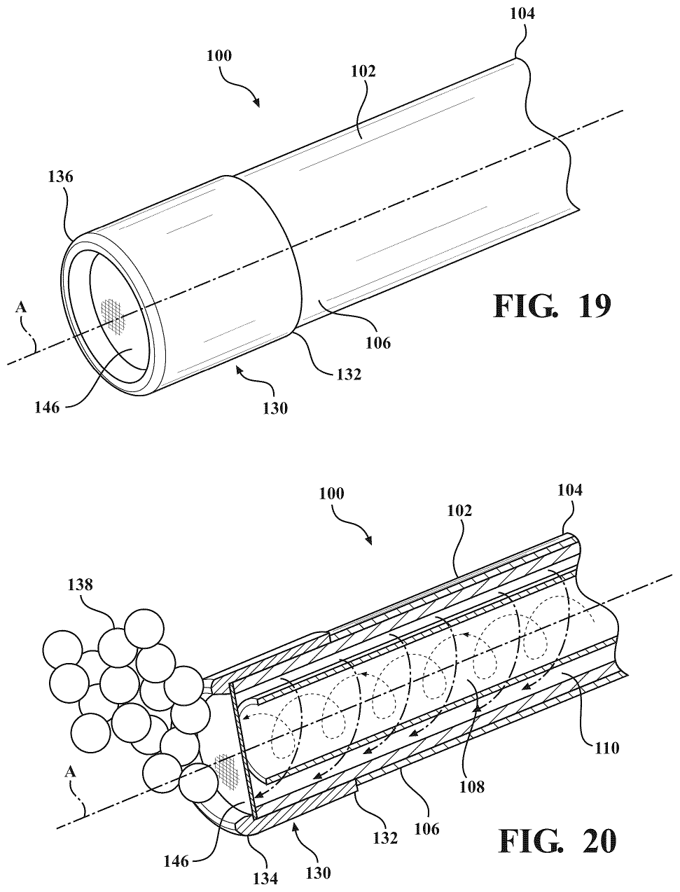

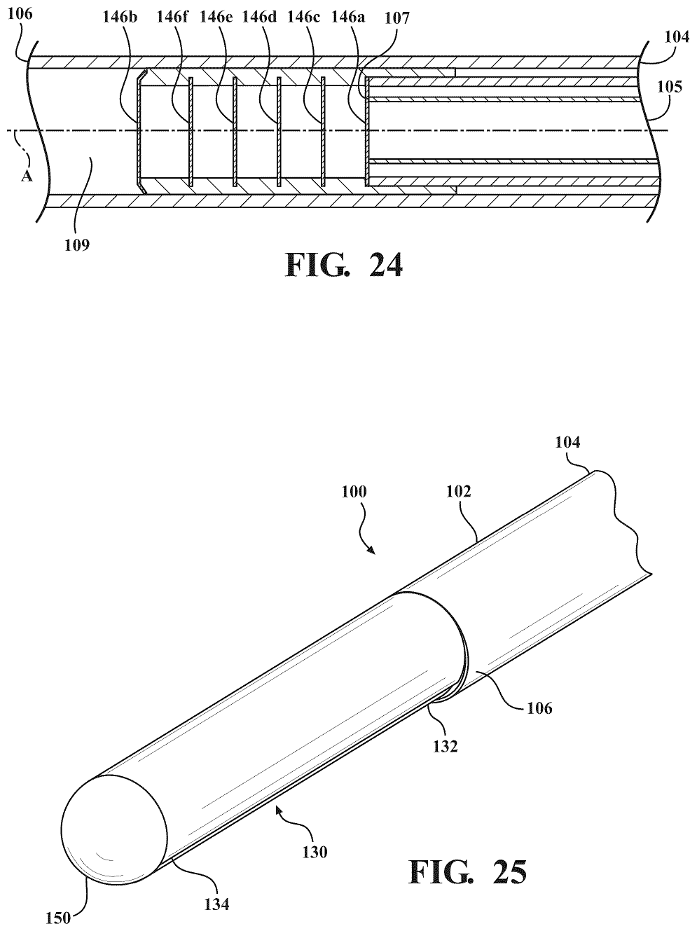

As illustrated in FIGS. 19-24, in accordance with a fourth embodiment, the foam generation assembly 130 includes at least one mesh disc 146 arranged transverse to a longitudinal axis A of the catheter shaft 12 between the entrance end 132 and the exit end 134. More preferably, as best shown in FIGS. 23-24, the foam generation assembly 130 includes a plurality of mesh discs 146a, 146b, 146c, 146d, 146e, 146f sequentially arranged between the entrance and exit ends 132, 134 to establish the plurality of shearing structures 136 through which the substances pass to generate the foam 138. Each pair of adjacent mesh discs 146 define a bubble chamber 148 extending therebetween to store any foam 138 generated from mixing the first and second substances as they pass through the initial mesh disc 146a for each bubble chamber 148. Additionally, each of the plurality of mesh discs 146 can have a porosity sized to optimize foam generation of the drug/solution in the bubble chamber 148 and out of the last mesh disc 146b disposed adjacent the exit end 136. Furthermore, the plurality of mesh discs 146 can be arranged incrementally from a coarse mesh disc 146a having a course mesh disposed next adjacent to the entrance end 132 to a fine mesh disc 146b having a fine mesh being finer than the course mesh and arranged next adjacent to the exit end 134 to force the first and second substances sequentially through respectively finer mesh discs 146, to create gross shear and generate foam 138 within the sequential bubble chambers 148 and out of the exit end 134 of the foam generation assembly 130.

As illustrated in FIGS. 25-27, in accordance with a fifth embodiment, the foam generation assembly 130 includes a rolled mesh 150 which is rolled concentrically around the longitudinal axis A of the catheter shaft 102 to define a plurality of circular mesh layers 152 (circular when viewed in a cross-sectional plane taken transverse to the longitudinal axis A) and sequentially arranged radially outwardly from the longitudinal axis A to an outermost mesh layer 152′. Each of the circular mesh layers 152 provide the sequentially arranged plurality of shearing structures 136 which mix the substances received from the plurality of lumens 108, 110 as they flow radially outwardly through the rolled mesh 150 and are conveyed through the plurality of circular mesh layers 152 to create gross shear and generate the foam 138 (See FIG. 27). Similar to the mesh disc embodiment, the rolled mesh 150 may have a porosity sized to optimize foam generation of the drug/therapy solution.

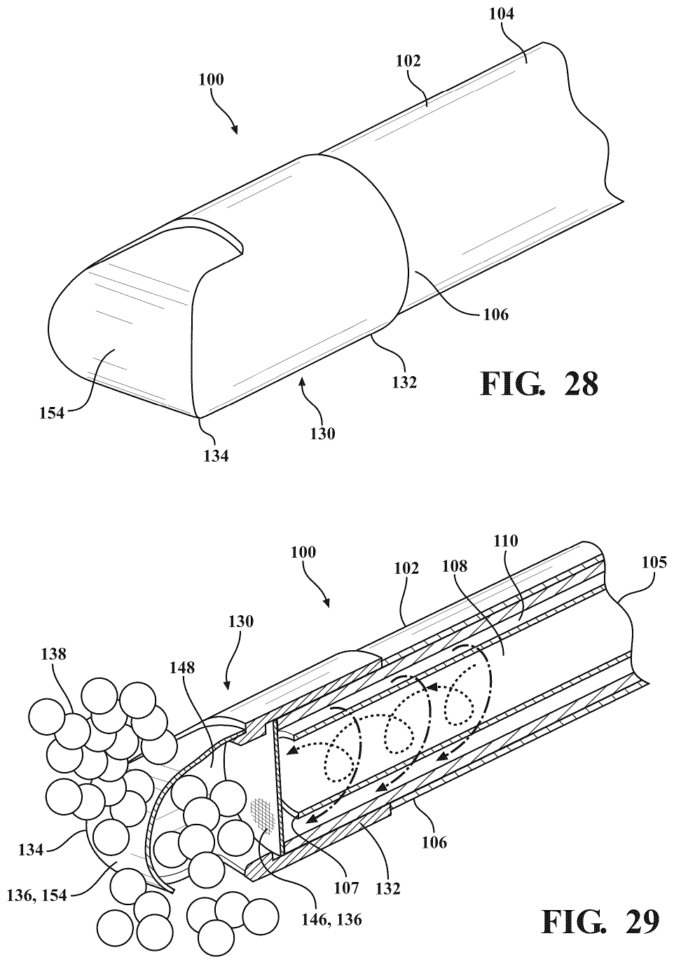

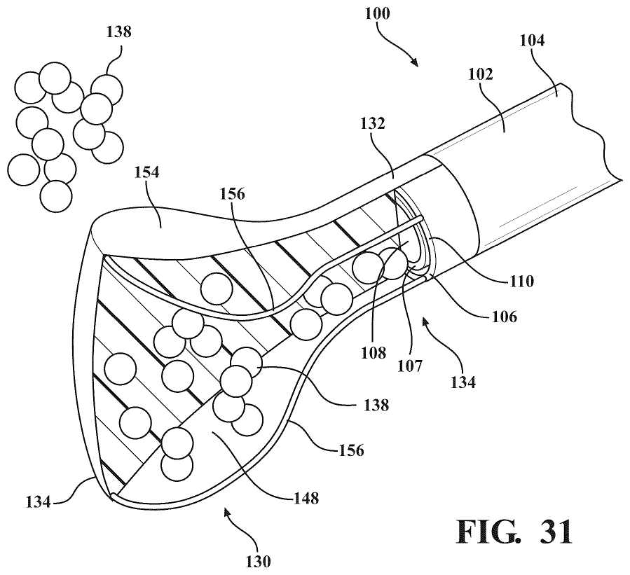

As illustrated in FIGS. 28-31, in a sixth embodiment, and similar to the fourth embodiment, the foam generation assembly 130 includes and houses a mesh disc 146 arranged transverse to a longitudinal axis of the catheter shaft 102 and between the entrance end 132 and the exit end 134 to establish a first shearing structure 136. However, as a variation on the fourth embodiment, the sixth embodiment of the foam generation assembly 130 arranges a three-dimensional (3D) filter tip 154 having a three-dimensional cross-sectional shape on the exit end 134 which is comprised of a collapsible mesh/porous body having a porosity similar to the mesh disc 146 as a sequential shearing structure 136 to create gross shear and generate foam 138 as the substances sequentially pass through the mesh disc 146 and then the 3D filter tip 154. (See FIGS. 29 and 31). Similar to the fourth embodiment, a bubble chamber 148 extends between the mesh disc 146 and the 3D filter tip 154 to store any foam 138 generated from mixing the first and second substances as they pass through the initial mesh disc 146.

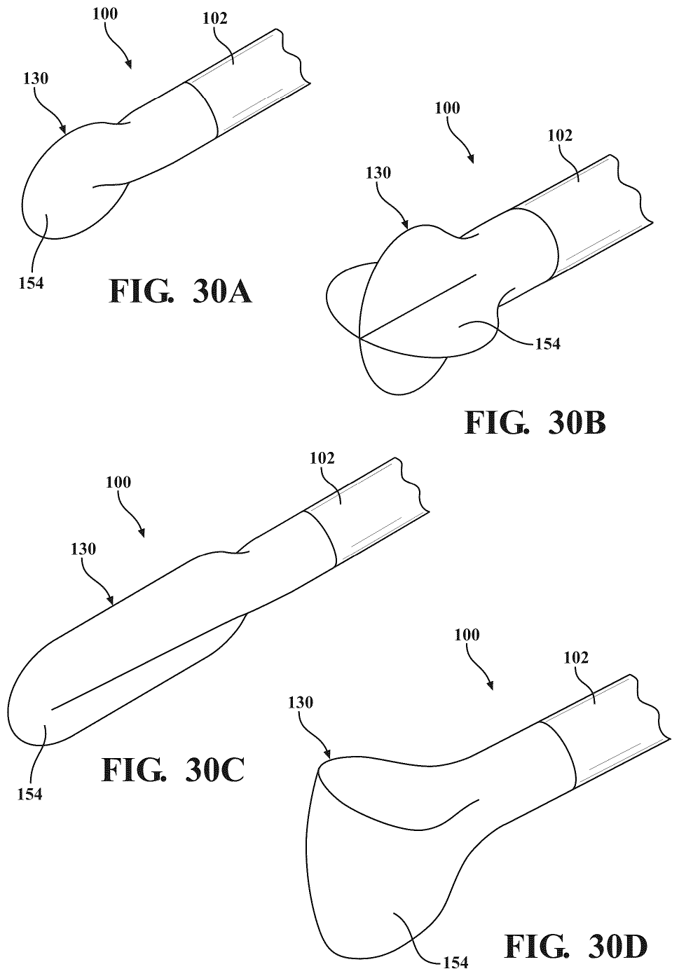

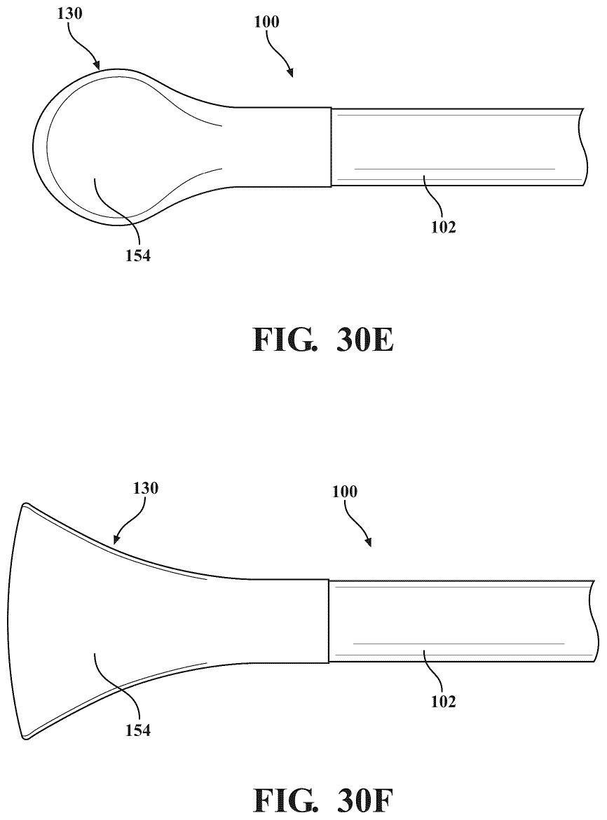

As best illustrated in FIGS. 30A-F, the 3D filter tip 154 can have various geometries and shapes depending on the intended application and the restricted channel through which the filter tip 154 must pass. Similar to the first, second and third embodiments, the mesh/porous body of the 3D filter tip 154 may also be pliable such that the foam generation assembly 130 may be compressed when passing through a restricted channel such as an endoscope instrument channel or other means of delivering the catheter 100. After being compressed, the 3D filter tip 154 of the foam generation assembly 130 may expand to its original size or shape. As best illustrated in FIG. 31, the 3D filter tip 154 can include at least one strut 156 (e.g., a wire form, which is independent of or part of the catheter shaft 102) to define the three-dimensional profile and different geometries of the 3D filter tip 154. The at least one strut 156 is collapsible with the mesh/porous body of the 3D filter tip 154 (such as when the foam generation assembly 130 is compressed when passing through the restricted channel) but then assists in returning and expanding the 3D filter tip 154 back to its original size and shape.

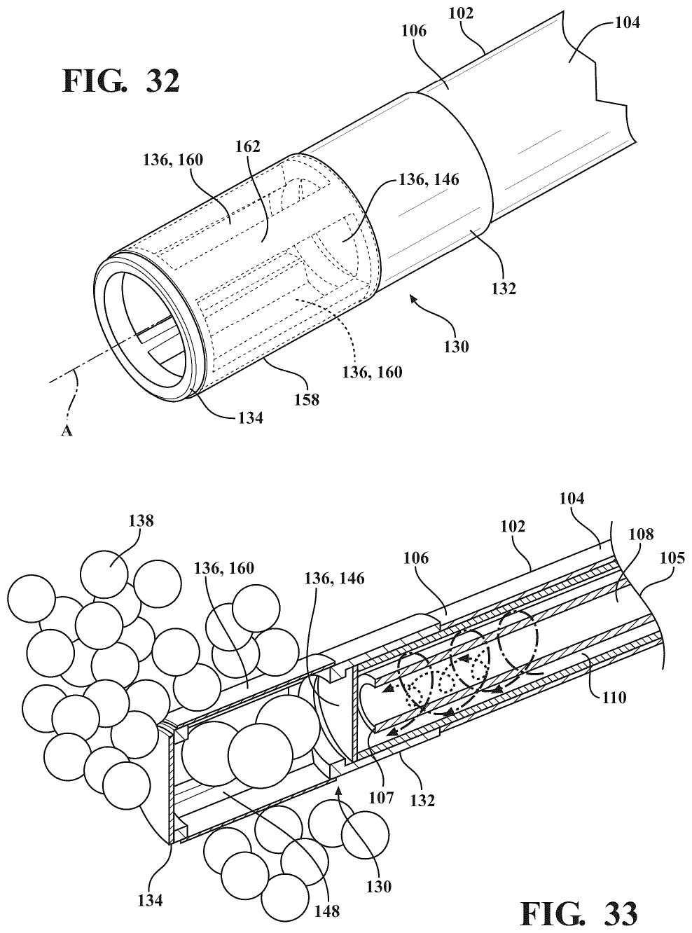

As illustrated in FIGS. 32-33, in a seventh embodiment, and similar to the sixth embodiment, the foam generation assembly 130 includes and houses a mesh disc 146 (best shown in FIG. 33) arranged transverse to the longitudinal axis A of the catheter shaft 12 and between the entrance end 132 and the exit end 134 as a first shearing structure 136. However, as a variation on the sixth embodiment, the seventh embodiment of the foam generation assembly 130 arranges a paneled filter tip 158 on the exit end 134 which is tubular in shape and presents a plurality of mesh panels 160 arranged concentrically around and in radially spaced relationship with the longitudinal axis A as additional shearing structures 136. A single mesh panel 160 could also be arranged continuously around the longitudinal axis A without departing from the scope of the subject disclosure. However, in either arrangement, the mesh panel(s) 160 are comprised of a mesh/porous body having a porosity similar to the mesh disc 146 to create gross shear and generate foam 138 as the substances sequentially pass through the initial mesh disc 146 and then the mesh panel(s) 160 of the paneled filter tip 154. (See FIG. 33). As best illustrated in FIG. 32, at least one support member 162 can extend along the mesh panel(s) 160 to provide structural support for the mesh/porous body of the mesh panel(s) 160. Similar to the sixth embodiment, and as best illustrated in FIG. 33, a bubble chamber 148 extends between the mesh disc 146 and the paneled filter tip 158 to store any foam 138 generated from mixing the first and second substances as they pass through the initial mesh disc 146.

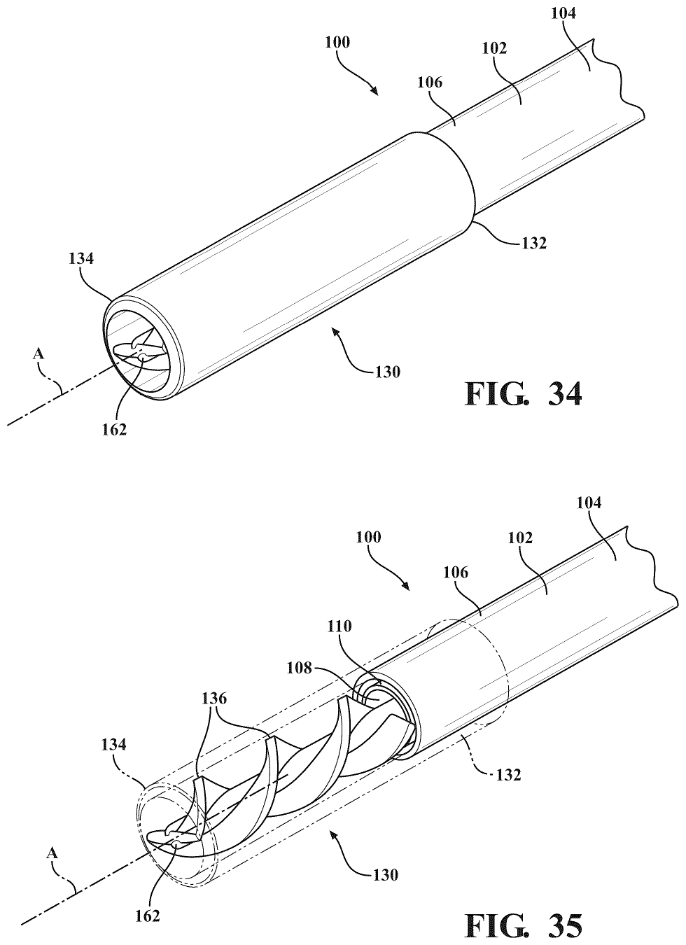

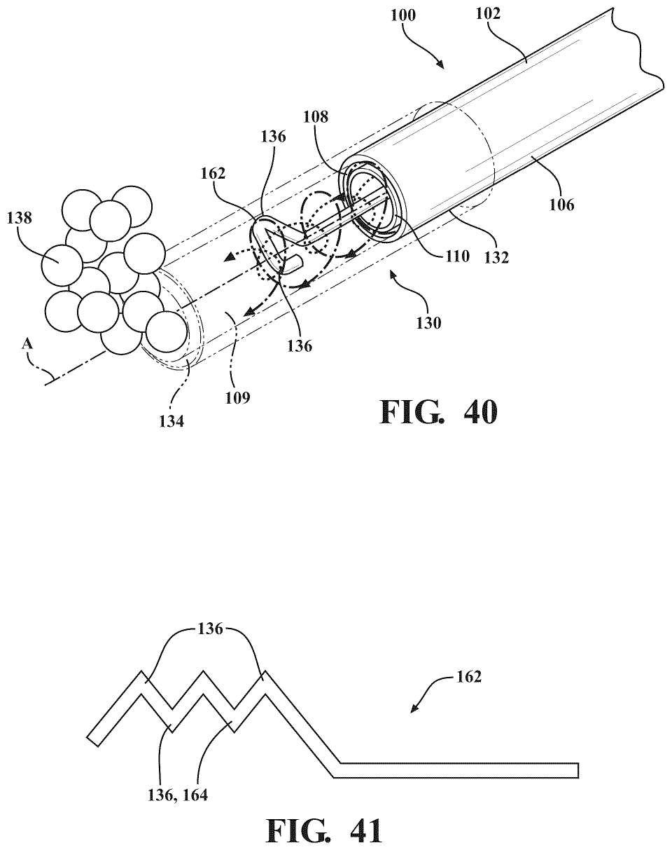

As illustrated in FIGS. 34-40, in an eighth embodiment, the foam generation assembly 130 includes an impeller 162 extending between the entrance and exit ends 132, 134 and which is rotatable about the longitudinal axis A of the catheter shaft 102. As best illustrated in FIGS. 34-39, in accordance with a first arrangement, the impeller 162 is threaded to present a plurality of shearing structures 136 arranged along the impeller between the entrance and exit ends 132. However, as best illustrated in FIGS. 40-41, in an alternative arrangement, the impeller 162 can be comprised of an eccentric wire form which is non-symmetrical relative to the longitudinal axis A to present a plurality of shearing structures 136 by way of an offset arrangement and vibration created during rotation of the impeller 162 about the axis. For example, as illustrated in FIG. 40, the eccentric wire form can have a hook-like cross-sectional shape that presents various shearing structures 136. Alternatively, as illustrated in FIG. 41, the eccentric wire form can have multiple bends 161 arranged offset relative to the longitudinal axis A to present the shearing structures 136. Such illustrations are exemplary, and other off-set arrangements of the eccentric wire form can be utilized without departing from the scope of the subject disclosure. In either of the aforementioned arrangements, rotation of the impeller 162 mixes the first substance (e.g., liquid drug solution) and the second substance (e.g., CO₂ gas) entering the foam generation assembly 130 from the plurality of lumens 108, 110 and creates gross shear to generate foam 138 (See FIGS. 29-30) at the exit end 134.

Obviously, many modifications and variations of the present invention are possible in light of the above teachings and may be practiced otherwise than as specifically described while within the scope of the appended claims.

What is claimed is:

1. A catheter comprising:
a catheter shaft extending along a longitudinal axis from a proximal catheter end to a distal catheter end;
a plurality of lumens housed with said catheter shaft and each configured to separately convey respective substances from a proximal lumen end disposed adjacent said proximal catheter end to a distal lumen end;
a foam generation assembly extending from an entrance end fluidly connected to said distal lumen ends of said plurality of lumens for receiving the respective substances to an exit end disposed adjacent or in spaced relationship with said distal catheter end of said catheter shaft; and
said foam generation assembly including a plurality of shearing structures arranged between said entrance and exit ends for mixing the respective substances as they are conveyed through said foam generation assembly to generate a foam at said exit end.

2. The catheter as set forth in claim 1, wherein said distal lumen ends terminate adjacent said distal catheter end and said foam generation assembly is disposed adjacent said distal catheter end of said catheter shaft.

3. The catheter as set forth in claim 1, wherein said distal lumen ends terminate in spaced relationship with said distal catheter end to define a mixing chamber extending therebetween, and said foam generation assembly is arranged within said catheter shaft in spaced relationship with said distal catheter end to dispose said exit end in fluid communication with said mixing chamber.

4. The catheter as set forth in claim 1, wherein said foam generation assembly is comprised of an open cell foam body having a lattice of interconnected cells or pores within said open cell foam body to define said plurality of shearing structures.

5. The catheter as set forth in claim 4, wherein said foam generation assembly is arranged on said distal catheter end of said catheter shaft and said open cell foam body has a polygonal cross-sectional shape.

6. The catheter as set forth in claim 4, wherein said foam generation assembly is disposed within said catheter shaft in spaced relationship with said distal catheter end and said open cell foam body has a cylindrical cross-sectional shape.

7. The catheter as set forth in claim 1, wherein said foam generation assembly is comprised of at least one layer of porous woven material having a lattice of sequentially arranged pores to define said plurality of shearing structures.

8. The catheter as set forth in claim 1, wherein said foam generation assembly is comprised of at least one layer of fibrous woven material having a lattice of sequentially arranged fibers to define said plurality of shearing structures.

9. The catheter as set forth in claim 1, wherein said foam generation assembly includes at least one mesh disc arranged transverse to said longitudinal axis of said catheter shaft to establish at least one of said plurality of shearing structures.

10. The catheter as set forth in claim 9, wherein said foam generation assembly includes a plurality of mesh discs sequentially arranged between said entrance and exit ends to establish said plurality of shearing structures.

11. The catheter as set forth in claim 10, wherein said plurality of mesh discs are arranged incrementally from an initial mesh disc disposed next adjacent to said entrance end and having a course mesh to a final mesh disc disposed next adjacent to said exit end and having a fine mesh being finer than said course mesh of said initial mesh disc.

12. The catheter as set forth in claim 9, wherein said at least one mesh disc is disposed adjacent said entrance end and said foam generation assembly further includes a three-dimensional filter tip having a three-dimensional cross-sectional shape disposed adjacent said exit end to establish an additional one of said plurality of shearing structures.

13. The catheter as set forth in claim 12, wherein said three-dimensional filter tip includes at least one strut to define and maintain the three-dimensional cross-sectional shape.

14. The catheter as set forth in claim 9, wherein said at least one mesh disc is disposed adjacent said entrance end and said foam generation assembly further includes a paneled filter tip disposed adjacent said exit end and being tubular in shape to present a plurality of mesh panels arranged concentrically around and in radially spaced relationship with said longitudinal axis to establish additional ones of said plurality of shearing structures.

15. The catheter as set forth in claim 14, wherein said paneled filter tip includes at least one support member extending along and providing structural support to said plurality of mesh panels.

16. The catheter as set forth in claim 1, wherein said foam generation assembly includes an impeller extending between said entrance and exit ends and being rotatable about said longitudinal axis of said catheter shaft.

17. The catheter as set forth in claim 16, wherein said impeller is threaded to present said plurality of shearing structures arranged along said impeller between said entrance and exit ends.

18. The catheter as set forth in claim 16, wherein said impeller is comprised of an eccentric wire form being non-symmetrical relative to said longitudinal axis to present said plurality of shearing structures.

19. The catheter as set forth in claim 18, wherein said eccentric wire form having a hook-like cross-sectional shape.

20. The catheter as set forth in claim 1, wherein said foam generation assembly includes a rolled mesh being rolled concentrically around said longitudinal axis of said catheter shaft to define a plurality of circular mesh layers sequentially arranged radially outwardly from said longitudinal axis to establish said plurality of shearing structures.
